(19) United States
(12) Patent Application Publication
(10) Pub. No.: US 2019/0388101 A1
(43) Pub. Date: Dec. 26, 2019

(54) Tibia Files and Associated Kits and Methods

(71) Applicant: Richard E. Grant, Ambler, PA (US)

(72) Inventor: Richard E. Grant, Ambler, PA (US)

(21) Appl. No.: 16/379,756

(22) Filed: Apr. 9, 2019

Related U.S. Application Data
(60) Provisional application No. 62/690,004, filed on Jun. 26, 2018.

(51) Int. Cl. A61B 17/16

(52) U.S. Cl. CPC A61B 17/1659; A61B 17/157; A61B 17/1675

(57) ABSTRACT

Medical devices for filing bones are described. A bone file has a kidney-shaped main body and a handle. The kidney-shaped main body has a leading recess that extends between first and second arm portions and a first surface that defines a kidney-shaped filing surface. A handle is continuous with the main body and extends away from the leading recess. The handle has a handle end portion opposite the leading recess that defines an aperture.

### FIELD

The disclosure relates to the field of medical instruments. More particularly, the disclosure relates to the field of bone files.

### BACKGROUND

Various tibia files for use during orthopedic knee surgery are known in the art. Known files, however, have several drawbacks. For example, bone files typically used in knee surgery only have a filing surface for one condyle surface of the knee, which often necessitates separate filing actions on the separate condyle surfaces. In turn, the use of separate filing actions can increase the likelihood that even filing is not achieved on the condyles. Furthermore, when uneven filing does occur, known tibia files typically used in knee surgery provide an ineffective adjustment tool for correcting the uneven filing.

A need exists, therefore, for improved tibia files, kits, and methods.

### BRIEF SUMMARY OF SELECTED EXAMPLES

Various example tibia files, kits, and methods are described herein.

An example tibia file includes a kidney-shaped main body and a handle. The kidney-shaped main body has first and second arm portions and first and second opposing surfaces. The main body defines a leading recess extending between the first and second arm portions and the first surface defines a kidney-shaped filing surface. A handle is continuous with the main body and extends away from the leading recess. The handle has a handle body and a handle end portion opposite the leading recess. The handle end portion defines an aperture.

Another example tibia file includes a kidney-shape main body and a handle. The kidney-shaped main body has first and second arm portions and first and second opposing surfaces. The main body defines a leading recess extending between the first and second arm portions and the first surface defines a kidney-shaped filing surface. The first surface lies in a first plane and a second surface lies in a second plane where the first and second planes are not parallel. The first surface is disposed on a first plane that forms a first angle relative to the lateral axis of the main body. A handle is continuous with the main body and extends away from the leading recess. The handle has a handle body and a handle end portion opposite the leading recess. The handle end portion defines an aperture.

Another example tibia file includes a kidney-shape main body and a handle. The kidney-shaped main body has first and second arm portions and first and second opposing surfaces. The main body defines a leading recess extending between the first and second arm portions and the first surface defines a kidney-shaped filing surface. The first surface lies in a first plane and a second surface lies in a second plane where the first and second planes are not parallel. The first surface is disposed on a first plane that forms a second angle relative to the lateral axis of the main body. A handle is continuous with the main body and extends away from the leading recess. The handle has a handle body and a handle end portion opposite the leading recess. The handle end portion defines an aperture.

An example kit includes a tibia file and a plumb member. The tibia file comprises a kidney-shaped main body and a handle. The kidney-shaped main body has first and second arm portions and first and second opposing surfaces. The main body defines a leading recess extending between the first and second arm portions and the first surface defines a kidney-shaped filing surface. The handle is continuous with the main body and extends away from the leading recess. The handle has a handle body and a handle end portion opposite the leading recess. The handle end portion defines an aperture. Furthermore, the plumb member comprises a stop member attached to a rod member.

An example method of treatment includes a step of introducing a first tibia file on the tibial condyle surfaces. The first tibia file includes a kidney-shaped main body and a handle. The kidney-shaped main body has first and second arm portions and first and second opposing surfaces. The main body defines a leading recess extending between the first and second arm portions and the first surface defines a kidney-shaped filing surface. A handle is continuous with the main body and extends away from the leading recess. The handle has a handle body and a handle end portion opposite the leading recess. The handle end portion defines an aperture. The first surface lies in a first plane and a second surface lies in a second plane where the first and second planes are parallel. Another step includes advancing a first tibia file onto the condyle tibial surfaces to be drawn back and forth parallel to the bone surface to facilitate bone filing. Another step includes inserting a plumb component into an aperture of the handle end portion of the first tibia file. The plumb member includes a stop member attached to a rod member. The aperture interfaces with the stop member of the plumb. The aperture is sized and configured to receive the rod member of a plumb. Another step includes confirming the condyle tibial surfaces are aligned perpendicular to the axis of the tibia such that the plumb member is aligned parallel to the axis of the tibia.

### DESCRIPTION OF FIGURES

FIG. 1 is a top view of an example tibia file.

FIG. 2 is a side view of the example tibia file illustrated in FIG. 1.

FIG. 3 is an end view of the example tibia file illustrated in FIG. 1.

FIG. 4 is a top view of another example tibia file.

FIG. 5 is a perspective view of another example tibia file and a plumb member.

FIG. 6 is a side view of the example tibia file and the plumb member illustrated in FIG. 5. The plumb member is disposed through the aperture of the tibia file.

FIG. 7 is a perspective view of another example tibia file.

FIG. 8 is an end view of the example tibia file illustrated in FIG. 7.

FIG. 9 is a perspective view of another example tibia file.

FIG. 10 is an end view of the example tibia file illustrated in FIG. 9.

FIG. 11 illustrates an example kit.

FIG. 12 is a flowchart representation of an example method of treatment.

### DESCRIPTION OF SELECTED EXAMPLES

The following detailed description and the appended drawings describe and illustrate various tibia files, kits, and methods. The description and drawing are provided to enable one skilled in the art to make and use one or more tibia files, kits, and/or practice one or more methods. They are not intended to limit the scope of the claims in any manner.

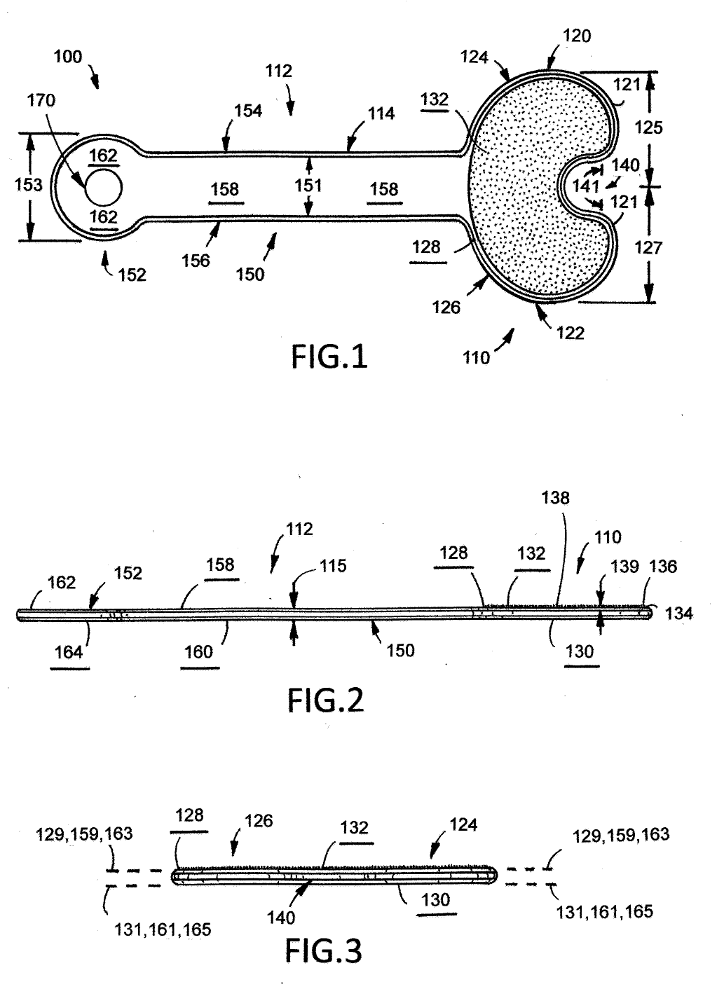

FIGS. 1, 2, and 3 illustrate a first example tibia file 100. The tibia file 100 has a kidney-shaped main body 110, a handle 112, and a wall 114.

The kidney-shaped main body 110 has a main body first end 120, a main body second end 122, a first arm portion 124, a second arm portion 126, a first surface 128, a second surface 130, and a leading recess 140.

The first arm portion 124 has a first arm portion first length 125 that extends between the main body first end 120 towards the medial portion of the main body 110. The second arm portion 126 has a second arm portion second length 127 that extends between the main body second end 122 towards the medial portion of the main body 110. As illustrated in FIG. 1, the first arm portion first length 125 and the second arm portion second length 127 are equal lengths and define the total length of the main body 110.

As illustrated in FIGS. 2 and 3, the first and second surfaces 128, 130 of the main body 110 are opposing surfaces. The first surface 128 defines a continuous, uninterrupted surface that extends between the first arm portion 124 and the second arm portion 126. The first surface 128 also defines a kidney-shaped filing surface 132 that defines a continuous, uninterrupted surface extending between the first arm portion 124 and the second arm portion 126. The second surface 130 defines a continuous, uninterrupted surface extending between the first arm portion 124 and the second arm portion 126. The first surface 128 and filing surface 132 lie in a first plane 129, and the second surface 130 lies in a second plane 131. As illustrated in FIG. 3, the first plane 129 and the second plane 131 lie in parallel due to both the first plane 129 and the second plane 131 running parallel to the lateral axis of the main body 110.

The filing surface 132 is a roughened surface suitable for filing bone. The filing surface can be roughened with a regular pattern, an irregular pattern, an added material, or other features suitable for constructing a roughened surface to file a bone. Furthermore, the roughened pattern of the filing surface can include a plurality of grooves, points, bumps, or other features suitable for a roughened pattern to file a bone.

The filing surface 132 of the first surface 128 includes a filing surface first end 134, and filing surface second end 136, and a filing element 138. The filing element 138 extends between the first arm portion 124 and the second arm portion 126. As illustrated in FIG. 2, the filing element 138 defines a filing element height 139 that extend from the filing surface first end 134 to the filing surface second end 136. In addition, as discussed above, the filing surface 132 and the filing element 138 lie in the first plane 129 that is parallel with the second surface 130.

As illustrated in FIG. 1, the filing surface 132 defines a kidney shape. The kidney shape allows the filing surface 132 to be positioned on the lateral tibial condyle and medial tibial condyle simultaneously. With this kidney shape, the filing surface 128 interfaces with the tibia and facilitates bone filing on the lateral tibial condyle and the medial tibial condyle simultaneously. In addition, the kidney shape is considered advantageous for a filing surface at least because this allows for coinciding bone filing between the lateral tibial condyle and the medial tibial condyle, which can decrease the use of bone filing during a knee surgery.

In the illustrated embodiment, the first surface and second surface 128, 130 is formed of a first material and the filing surface 132 is formed of a second material. In one example, the first material comprises a first material property and the second material comprises a second material property. Here, the first and second materials comprise different material properties such as different types of metals. Alternatively, the material of the first and second surfaces is formed of a first material that is the same as the second material that forms the filing surface. Thus, the first and second materials have identical material properties. Skilled artisans will be able to select suitable materials to construct the first and second surfaces and the filing surface according to a particular embodiment based on various considerations, including materials that are advantageous for proper bone removal and are non-corrosive.

The tibia file 100, and, therefore, the first and second surfaces 128, 130, can be made from any conventional material. Skilled artisans will be able to select a suitable material or materials to construct a tibia file according to a particular embodiment based on various considerations, including any handling, processing, and performance preferences. Examples of suitable materials for both surfaces include stainless steel and other metals and alloys commonly used in medical devices and equipment.

The leading recess 140 is defined by a curvilinear edge 121 of the main body 110. The leading recess 140 has a leading recess first length 141 that is measured along curvilinear edge 121 of the main body 110 from the first arm portion 124 to the second arm portion 126. The leading recess 140 is sized and configured to accommodate a posterior crucial ligament during filing use. By accommodating the posterior crucial ligament, the leading recess 140 allows the tibia file 100 to be drawn back and forth parallel to the bone surface in an anterior-posterior direction or in a lateral-medial direction to facilitate bone filing without contacting the posterior crucial ligament during knee surgery.

The handle 112 is continuous with the main body 110 and extends away from the leading recess 140. The handle 112 has a handle body 150 and a handle end portion 152 opposite the leading recess 140. The handle body 150 defines a handle body first width 151, and the handle end portion 152 defines a handle end portion second width 153. As illustrated in FIG. 1, the handle end portion second width 153 is greater than the handle body first width 151.

The handle body 150 has a handle body first end 154, a handle body second end 156, a handle body length 155, a third surface 158, and a fourth surface 160. The handle body length 155 extends between the main body 110 towards the handle end portion 152. As illustrated in FIG. 2, the third and fourth surfaces 158, 160 are opposing surfaces. The third surface 158 defines a continuous, uninterrupted surface that extends between the handle body first end 154 and the handle body second end 156. The third surface 158 is disposed adjacent to the first surface 128 of the filing surface 132 of the main body 110. The fourth surface 160 defines a continuous, uninterrupted surface that extends between the handle body first end 154 and the handle body second end 156. The fourth surface 160 is disposed adjacent to the second surface 130 of the main body 110. The third surface 158 lies in a third plane 159 and the fourth surface 160 lies in a fourth plane 161. As illustrated in FIG. 2, the third plane 159 and the fourth plane 161 lie in parallel due to both the third plane 159 and the fourth plane 161 running parallel to the lateral axis of the handle body 150.

The handle end portion 152 has a fifth surface 162, a sixth surface 164, and an aperture 170. As illustrated in FIG. 2, the fifth and sixth surfaces 162, 164 are opposing surfaces. The fifth surface 162 defines a continuous, uninterrupted surface that extends circumferentially from the aperture 170. The fifth surface 162 is disposed adjacent to the third surface 158 of the handle body 150. The sixth surface 164 defines a continuous, uninterrupted surface that extends circumferentially from the aperture 170. The sixth surface 164 is disposed adjacent to the fourth surface 160 of the handle body 150. The fifth surface 162 lies in a fifth plane 163 and the sixth surface 164 lies in a sixth plane 165. As illustrated in FIG. 2, the fifth plane 163 and the sixth plane 165 lie in parallel due to both the fifth plane 163 and the sixth plane 165 running parallel to the lateral axis of the handle end portion 152. The aperture 170 of the handle end portion 152 is disposed opposite from the leading recess 140. The aperture 170 is disposed in the center of the handle end portion 152 and extends between the fifth surface 162 and the sixth surface 164 of the handle end portion 152.

The wall 114 lies on a plane that is vertical to the first, second, third, fourth, fifth, and sixth planes 129, 131, 159, 161, 163, 165. The wall 114 extends circumferentially from the main body 110 and away from the leading recess 140 towards the handle body 150 and the handle end portion 152. The wall 114 also defines a wall width 115. The wall width 115 is collectively defined by extending between the first and second surfaces 128, 130, the third and fourth surfaces 158, 160, and the fifth and sixth surfaces 162, 164.

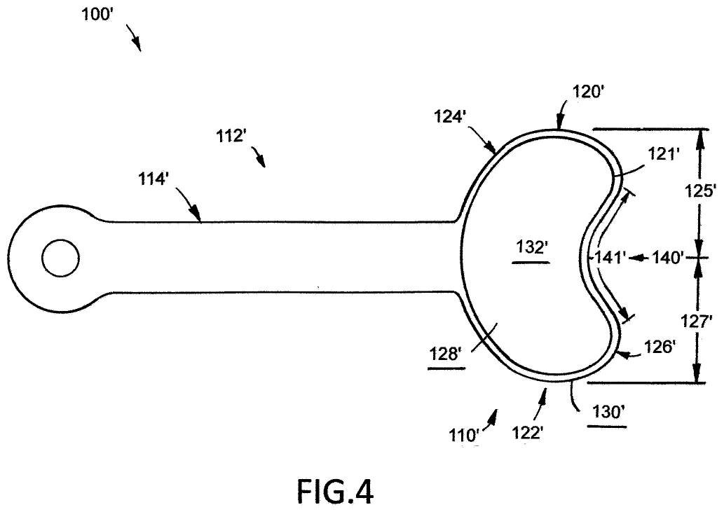

FIG. 4 illustrates another example tibia file 100′. The tibia file 100′ is similar to the tibia file 100 illustrated in FIGS. 1, 2, and 3 and described above, except as detailed below. The tibia file 100′ includes a kidney-shaped main body 110′, a handle 112′, and a wall 114′.

The kidney-shaped main body 110′ has a main body first end 120′, a main body second end 122′, a first arm portion 124′, a second arm portion 126′, a first surface 128′, a second surface 130′, and a leading recess 140′.

The first arm portion 124′ has a first arm portion first length 125′ that extends between the main body first end 120′ towards the medial portion of the main body 110′. The second arm portion 126′ has a second arm portion second length 127′ that extends between the main body second end 122′ towards the medial portion of the main body 110′. As illustrated in FIG. 4, the first arm portion first length 125′ and the second arm portion second length 127′ are equal lengths and define the total length of the main body 110′. In addition, the first arm portion first length 125′ of the second example tibia file 100′ is greater than the first arm portion first length 125 of the first example tibia file 100. The second arm portion second length 127′ of the second example tibia file 100′ is greater than the second arm portion second length 127 of the first example tibia file 100.

The leading recess 140′ is defined by a curvilinear edge 121′ of the main body 110′. The leading recess 140′ has a leading recess first length 141′ that is measured along curvilinear edge 121′ of the main body 110′ from the first arm portion 124′ to the second arm portion 126′. In the illustrated embodiment, the leading recess first length 141′ of the second example tibia file 100′ is greater than the leading recess first length 141 of the first example tibia file 100. The leading recess 140′ is an alternative to the leading recess 140 because the leading recess 140′ is also sized and configured to accommodate a posterior crucial ligament during filing use when described in the first example tibia file 100. In addition, the alternative leading recess 140′ is considered advantageous at least because it provides a structural arrangement that allows for an increased range of motion in the lateral-medial direction to facilitate bone filing without contacting the posterior crucial ligament.

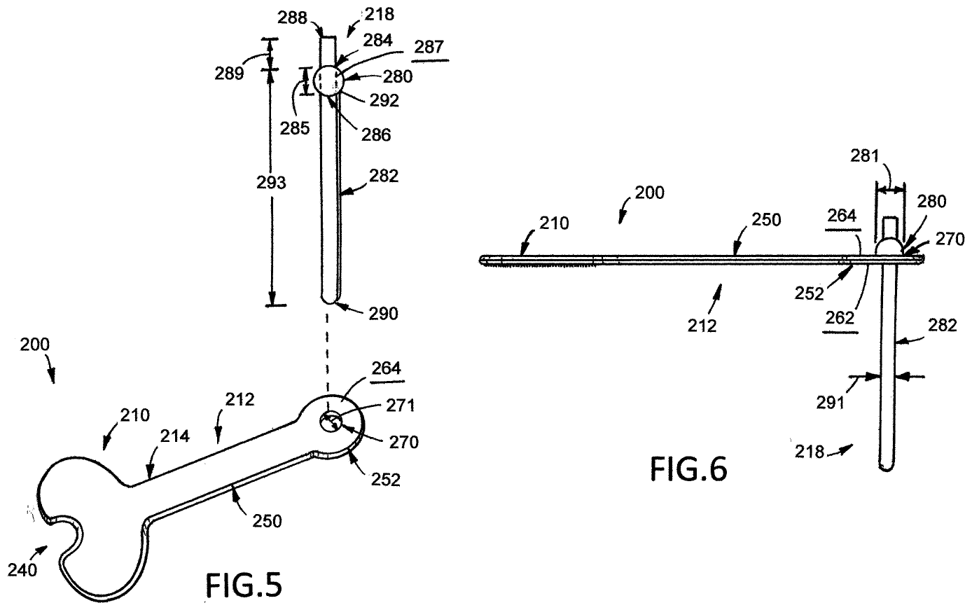

FIGS. 5 and 6 illustrate another example tibia file 200 and a plumb member 218. The tibia file 200 is similar to the tibia file 100 illustrated in FIGS. 1, 2, and 3 and described above, except as detailed below.

In the illustrated embodiment, the plumb member 218 has a stop member 280 and a rod member 282.

The stop member 280 has a stop member first side 284, a stop member second side 286, a stop member length 285, a stop member surface 287, and an opening 292. The stop member length 285 extends from the stop member first side 284 to the stop member second side 286. The stop member surface 287 extends circumferentially between the stop member first side 284 and the stop member second side 286. The opening 292 extends between the stop member first side 284 to the stop member second side 286. As illustrated in FIGS. 5 and 6, the stop member 280 comprises a rounded shape. A skilled artisan will be able to select an appropriate shape for a particular stop member. Example shapes considered suitable for the stop member include, but are not limited to, circular, semi-circular, ovoid, arcuate, curvilinear, and any other shape considered suitable for a particular application.

The rod member 282 has a rod member first end 288, a rod member second end 290, a rod member first width 291, a rod member first length 289, and a rod member second length 293. The rod member first length 289 extends from the rod member first end 288 to the stop member first side 284. The rod member second length 293 extends from the stop member first side 284 to the rod member second end 290. The rod member second length 293 is greater than the rod member first length 289. As illustrated in FIG. 5, the rod member first length 289 and the rod member second length 293 define the total length of the rod member 282.

The stop member 280 and the rod member 282 are attached at the stop member first side 284 and the stop member second side 286 due to the rod member 282 extending through the opening 292 of the stop member 282. A portion of the rod member 282 is disposed in the opening 292 of the stop member 280.

The tibia file 200 has a kidney-shaped main body 210, a handle 212, and a wall 214. The main body 210 and the wall 214 comprise the same description as the main body 110 and wall 114 of the first example tibia file 100. In addition, the handle 112 has a handle body 250 and a handle end portion 252 opposite the leading recess 240. The handle end portion 252 has a fifth surface 262, a sixth surface 264, an aperture 270, and an aperture second width 271. The fifth and sixth surfaces 262, 264 comprise the same description as the fifth and sixth surfaces 162, 164 of the first example tibia file 100 illustrated in FIGS. 1, 2, and 3.

As best illustrated in FIGS. 5 and 6, the aperture 270 is sized and configured to receive the rod member 282 of the plumb member 218. The aperture 270 is also configured to cooperatively interface with the stop member 280 of the plumb member 218. As described above, the rod member 282 has a rod member first width 291 and the aperture has an aperture second width 271. In the illustrated embodiment, the aperture second width 271 is greater than the rod member first width 291. The stop member 280 has a stop member third width 281 and the aperture has an aperture second width 271. In the illustrated embodiment, the stop member third width 285 is greater than the aperture second width 271. The structural arrangement between the aperture 270 and the plumb member 218 is considered advantageous because the plumb member 218 is held by the aperture 270 such that the stop member 280 and the rod member 282 do not move circumferentially on or inside the aperture 270. Thus, the structural arrangement allows the plumb member 218 and the tibia file 200 to cooperatively interface such that the structural arrangement measures the condyle tibial surfaces to the center of the tibia. For example, if the condyle tibial surfaces are not aligned perpendicular to the center of the tibia, the plumb member 218 will not be parallel to the axis of the tibia because the tibia file 200 is interfacing with non-parallel condyle tibial surfaces.

As illustrated in FIGS. 5 and 6, the rod member 282 extends through the opening 292 of the stop member 280. The rod member 282 extending through the stop member 280 is considered advantageous because this structural arrangement aids in an assessment of the alignment between the condyle tibial surfaces and the center of the tibia. For example, if the condyle tibial surfaces are not aligned perpendicular to the center of the tibia, the rod member 282 and the extension of the rod member 282 will not be parallel to the axis of the tibia because the tibia file 200 is interfacing with non-parallel condyle tibial surfaces.

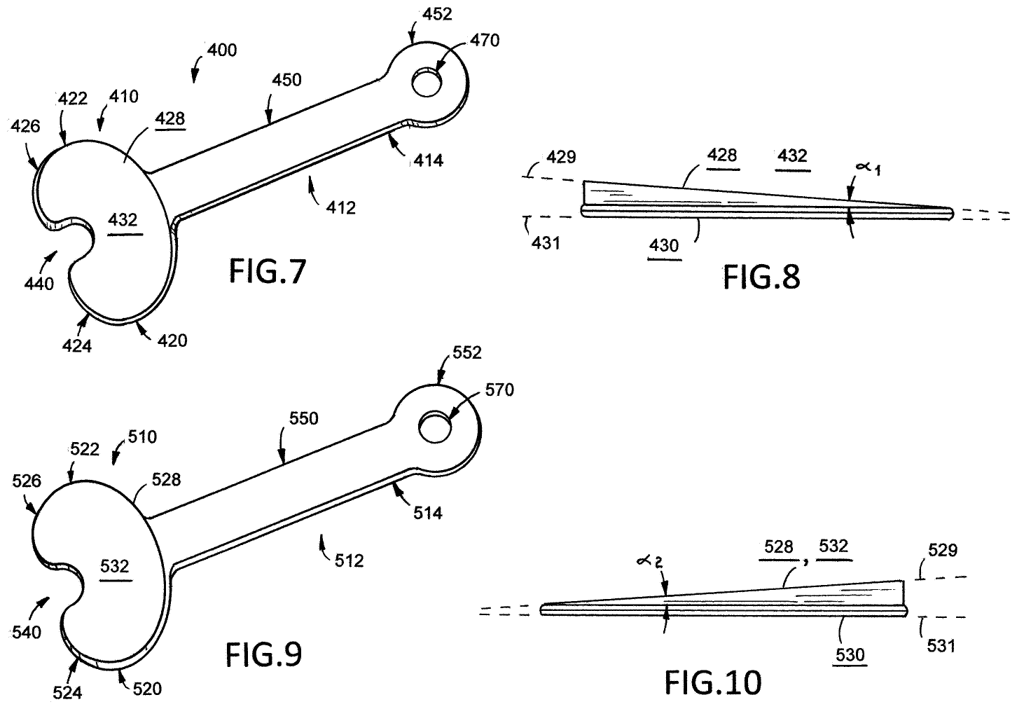

FIGS. 7 and 8 illustrate another example tibia file 400. The tibia file 400 is similar to the first example tibia file 100 illustrated in FIGS. 1, 2, and 3 and described above, except as detailed below.

The tibia file 400 has a kidney-shaped main body 410, a handle 412, and a wall 414. The kidney-shaped main body 410 has a main body first end 420, a main body second end 422, a first arm portion 424, a second arm portion 426, a first surface 428, a second surface 430, a filing surface 432, and a leading recess 440. The handle 412 is continuous with the main body 410 and extends away from the leading recess 440. The handle 412 has a handle body 450, and a handle end portion 452 and an aperture 470 opposite the leading recess 440.

In the illustrated embodiment, the first and second surfaces 428, 430 of the main body 410 are opposing surfaces. The first surface 428 and filing surface 432 lie in a first plane 429, and the second surface 430 lies in a second plane 431. As best illustrated in FIG. 8, the first plane 429 and the second plane 431 do not lie in parallel due to the first plane 429 not running parallel to the lateral axis of the main body 410. The first surface 428 and filing surface 430 are disposed on a first plane 429 that forms a first angle α₁ relative to the lateral axis of the main body 410. The first plane 429 of the first surface 428 and the filing surface 432 may define any suitable first angle α₁. A skilled artisan will be able to determine a suitable first angle for the first plane of the first surface and the filing surface according to a particular example based on various considerations, including the top surfaces of the lateral tibial condyle and the medial tibial condyle. Examples of suitable first angles include angles between about 1° and about 4° and an angle about 2°.

FIGS. 9 and 10 illustrate another example tibia file 500. The tibia file 500 is similar to the first example tibia file 100 illustrated in FIGS. 1, 2, and 3 and described above, except as detailed below.

The tibia file 500 has a kidney-shaped main body 510, a handle 512, and a wall 514. The kidney-shaped main body 510 has a main body first end 520, a main body second end 522, a first arm portion 524, a second arm portion 526, a first surface 528, a second surface 530, and a leading recess 540. The handle 512 is continuous with the main body 510 and extends away from the leading recess 540. The handle 512 has a handle body 550, and a handle end portion 552 and an aperture 570 opposite the leading recess 540.

In the illustrated embodiment, the first and second surfaces 528, 530 of the main body 510 are opposing surfaces. The first surface 528 and filing surface 532 lie in a first plane 529, and the second surface 530 lies in a second plane 531. As best illustrated in FIG. 10, the first plane 529 and the second plane 531 do not lie in parallel due to the first plane 529 not running parallel to the lateral axis of the main body 510. The first surface 528 and filing surface 530 are disposed on a first plane 529 that forms a second angle α₂ relative to the lateral axis of the main body 510. The first plane 529 of the first surface 528 and the filing surface 532 may define any suitable second angle α₂. A skilled artisan will be able to determine a suitable second angle for the first plane of the first surface and the filing surface according to a particular example based on various considerations, including the surfaces of the lateral tibial condyle and the medial tibial condyle. Examples of suitable second angles include angles between about 1° and about 4° and an angle about 2°.

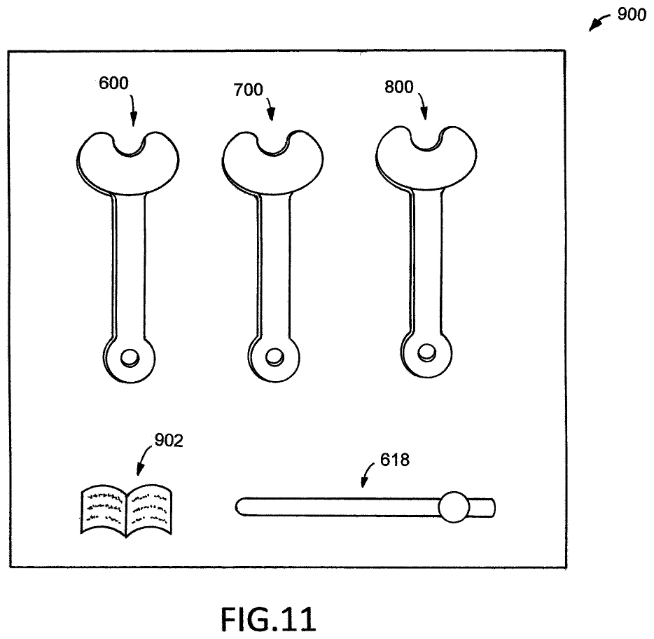

FIG. 11 illustrates an example kit 900 comprising a tibia file 600 according to an embodiment, such as tibia file 100, 100′, and 200 as illustrated and described herein; a second tibia file 700 according to an embodiment, such as tibia file 400 illustrated in FIGS. 7 and 8; a third tibia file 800 according to an embodiment, such as tibia file 500 illustrated in FIGS. 9 and 10; a plumb member 618 according to an embodiment, such as plumb member 218 illustrated in FIGS. 5 and 6; and instructions for use 902.

While the tibia files 600, 700, and 800 and plumb member 618 have been described and illustrated in kit 900, any suitable number of tibia files according to an embodiment and a plumb member according to an embodiment can be included in kit 900. Skilled artisans will be able to select a suitable number of tibia files and a plumb member according to a particular embodiment based on various considerations, including the surfaces of the lateral tibial condyle and the medial tibial condyle which a tibia file is intended to adjust. Example numbers of tibia files and a plumb member suitable to include in a kit include, but are not limited to one, two, three, four and any other number considered suitable for a particular application.

Furthermore, while tibia files 600, 700, and 800 and plumb member 618 have been described and illustrated in kit 900, any tibia file according to an embodiment and a plumb member according to an embodiment can be included in kit 900. Skilled artisans will be able to select a suitable tibia file and a plumb member according to a particular embodiment based on various considerations, including the surfaces of the lateral tibial condyle and the medial tibial condyle which a tibia file is intended to adjust. Example tibia files considered suitable to include in kit 900 include, but are not limited to, tibia file 100, tibia file 100′, tibia file 200, tibia file 400, tibia file 500, and/or any other tibia file considered suitable for a particular application. Moreover, an example plumb member considered suitable to include in kit 900 includes plumb member 218.

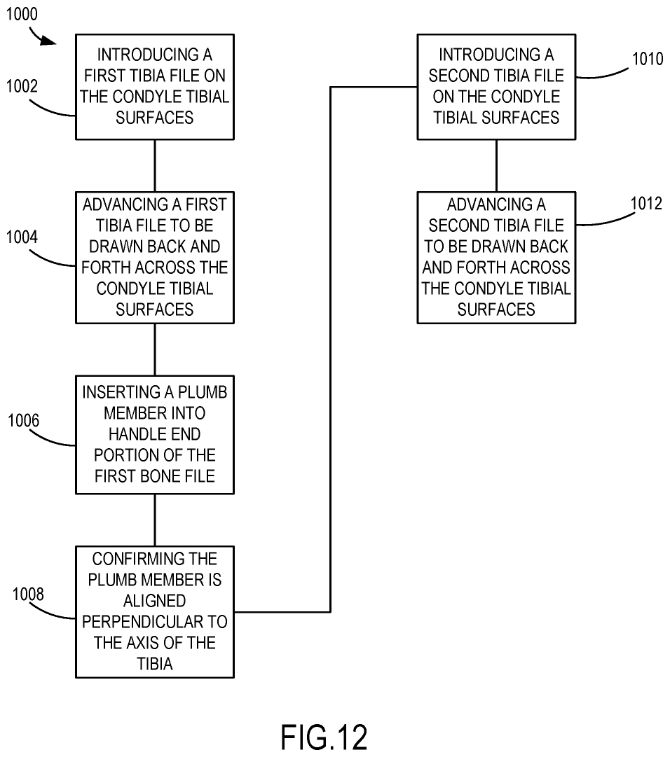

FIG. 12 is a flowchart representation of an example method 1000 of treatment for filing both condyle tibial surfaces with a tibia file. The initial step 1002 comprises introducing a first tibia file 100 on the condyle tibial surfaces such that the first tibia file 100 interfaces the condyle tibial surfaces. Another step 1004 comprises advancing the first tibia file 100 to be drawn back and forth parallel to the tibial condyle surface to facilitate bone filing of the condyle tibial surfaces. Another step 1006 comprises inserting a plumb member 218 into aperture 270 of the handle end portion 252 of the first tibia file 200. Another step 1008 comprises confirming the plumb member 218 is aligned parallel to the axis of the tibia.

The step 1002 of introducing a tibia file, such as a first tibia file 100, on the condyle tibial surfaces such that the tibia file interfaces with the condyle tibial surfaces can be accomplished using any suitable tibia file advancing a tibia file on the condyle tibial surface. While method 1000 has been described as using a first tibia file 100 to interface the condyle tibial surfaces, it is considered suitable to use any suitable tibia file on the condyle tibial surfaces. Skilled artisans will be able to select a suitable tibia file according to a particular embodiment based on various considerations, including the desired treatment intended to be performed. Example tibia files considered suitable include, but are not limited to, tibia file 100, tibia file 100′, tibia file 200, and any other suitable tibia file.

The step 1004 of advancing the tibia file 100, such as a first tibia file 100, to be drawn back and forth across the condyle tibial surface to facilitate bone filing of the condyle tibial surfaces can be accomplished by a surgeon drawing the first tibia file 100 in an anterior-posterior direction or in a medial-lateral direction. Furthermore, while method 1000 has been described as advancing the first tibia file 100 to be drawn back and forth to facilitate bone filing of the condyle tibial surfaces, it is considered suitable to use any suitable tibia file on the condyle tibial surface. Skilled artisans will be able to select a suitable tibia file according to a particular embodiment based on various considerations, including the desired treatment intended to be performed. Example tibia files considered suitable include, but are not limited to, tibia file 100, tibia file 100′, tibia file 200, and any other suitable tibia file according to an embodiment.

The step 1006 of inserting a plumb member 218 into the aperture 270 of the handle end portion 252 of the tibia file 200 can be accomplished by inserting the rod member 282 of the plumb member 218 into the aperture 270 on the sixth surface 264 of the handle end portion 252 of the tibia file 200. Furthermore, the stop member 280 of the plumb member 218 interfaces with the aperture 270 on the sixth surface 264 of the handle end portion 252 of the tibia file 200. Thus, the tibia file 200 and the plumb member 218 cooperatively interface to measure the condyle tibial surfaces relative to the axis of the tibia.

The step 1008 of confirming the condyle tibial surfaces are aligned perpendicular to the axis of the tibia such that the plumb member 218 is aligned parallel to the axis of the tibia can be accomplished by a surgeon observing the angle between the plumb member 218 and the axis of the tibia. The application of confirming that the condyle tibial surfaces are aligned perpendicular to the axis of the tibia allows the surgeon to determine if the condyle tibial surfaces need to be adjusted. If the condyle tibial surfaces is not aligned perpendicular to the axis of the tibia, steps 1002 through 1008 can be repeated a suitable number of times until the condyle tibial surface is aligned perpendicular to the axis of the tibia.

Various optional steps can be included, if desired. For example, if the lateral tibial condyle surface is not parallel to the medial tibial condyle surface or the medial tibial condyle surface is not parallel to the lateral tibial condyle surface, another step 1010 comprises introducing a second tibia file, such as the second tibia file 400 or the third tibia file 500, on the condyle tibial surfaces such that the second tibia file interfaces with the condyle tibial surfaces. Following the introduction of the tibia file, another step 1012 comprises advancing the second tibia file to be drawn back and forth across the non-parallel condyle tibial surfaces to facilitate bone filing of the condyle tibial surfaces in an anterior-posterior direction or in a medial-lateral direction. If steps 1010 and 1012 are performed, steps 1010 and 1012 are performed prior to steps 1006 and 1008. Furthermore, if steps 1010 and 1012 are performed, steps 1006 through 1012 can be repeated a suitable number of times until the lateral and the medial tibial condyle surfaces are parallel and are aligned perpendicular to the axis of the tibia.

The forgoing detailed description describes example tibia files, kits, and methods. The description and the appended drawing illustrating the described examples are intended only to provide examples and do not to limit the scope of the claims in any manner.

What is claimed is:

1. A bone file, comprising:
a kidney-shaped main body having first and second arm portions and first and second opposing surfaces, the main body defining a leading recess extending between the first and second arm portions, the first surface defining a kidney-shaped filing surface, the first surface lying in a first plane and the second surface lying in a second plane that is parallel to the first plane; and
a handle continuous with the main body and extending away from the leading recess, the handle having a handle body and a handle end portion opposite the leading recess, the handle end portion defining an aperture.

2. The bone file of claim 1, wherein the kidney-shaped main body has a curvilinear edge that defines the leading recess.

3. The bone file of claim 1, wherein the kidney-shaped filing surface defines a continuous, uninterrupted surface.

4. The bone file of claim 1, wherein the kidney-shaped filing surface is a roughened surface suitable for filing bone.

5. The bone file of claim 1, wherein each of the first surface and the second surface is formed of a first material and the kidney-shaped filing surface is formed of a second material.

6. The bone file of claim 5, wherein the first material and the second material are different.

7. The bone file of claim 5, wherein the first material and the second material are the same.

8. The bone file of claim 1, wherein the leading recess is sized and configured to accommodate a posterior crucial ligament.

9. The bone file of claim 1, wherein the handle includes a first width and the handle end portion includes a second width; and
wherein the second width is greater than the first width.

10. The bone file of claim 1, wherein the handle has a third surface that lies in a third plane and an opposing fourth surface that lies in a fourth plane;
wherein the third and fourth planes lie in parallel.

11. The bone file of claim 1, wherein the handle end portion has a fifth surface that lies in a fifth plane, an opposing sixth surface that lies in a sixth plane, and a center;
wherein the fifth and sixth planes lie in parallel; and
wherein the aperture is disposed in the center of the handle end portion and extends between the fifth surface and the sixth surface.

12. The bone file of claim 1, wherein the kidney-shaped main body has a main body first end and a main body second end,
wherein the first arm portion has a first arm portion first length that extends between the main body first end towards a medial portion of the main body;
wherein the second arm portion has a second arm portion second length that extends between the main body second end towards the medial portion of the main body; and
wherein the first arm portion first length and the second arm portion second length are equal.

13. A bone file, comprising:
a kidney-shaped main body having first and second arm portions and first and second opposing surfaces, the main body having a curvilinear edge defining a leading recess extending between the first and second arm portions, the first surface defining a kidney-shaped filing surface that is continuous and uninterrupted, the first surface lying in a first plane and the second surface lying in a second plane that is not parallel to the first plane; and
a handle continuous with the main body and extending away from the leading recess, the handle having a handle body and a handle end portion opposite the leading recess, the handle end portion defining an aperture.

14. The bone file of claim 14, wherein the first surface lies in the first plane at an angle that is between about one degree and about four degrees.

15. The bone file of claim 14, wherein the first surface lies in the first plane at an angle about two degrees.

16. A bone file apparatus, comprising a bone file and a plumb member:
the bone file comprising a kidney-shaped main body having first and second arm portions and first and second opposing surfaces, the main body having a curvilinear edge defining a leading recess extending between the first and second arm portions, the leading recess sized and configured to accommodate a posterior crucial ligament, the first surface defining a kidney-shaped filing surface, a handle continuous with the main body and extending away from the leading recess, the handle defining a handle body having third and fourth opposing surfaces and a handle end portion opposite the leading recess having fifth and sixth opposing surfaces, the handle end portion defining an aperture;
wherein the third surface lies in a third plane and the fourth surface lies in a fourth plane, the third plane and the fourth plane are parallel; and
wherein the fifth surface lies in a fifth plane and the sixth surface lies in a sixth plane, the fifth plane and the sixth plane are parallel;
the plumb member comprising a stop member attached to a rod member, the aperture of the handle end portion is sized and configured to receive the rod member and configured to interface with the stop member.

17. The bone file apparatus of claim 16, wherein the first surface lies in a first plane and the second surface lies in a second plane; and
wherein the first and second planes are parallel.

18. The bone file apparatus of claim 16, wherein the first surface lies in a first plane and the second surface lies in a second plane; and
wherein the first and second planes are not parallel.

19. The bone file apparatus of claim 18, wherein the first surface lies in the first plane at an angle that is between about one degree and about four degrees.

20. The bone file apparatus of claim 18, wherein the first surface lies in the first plane at an angle about two degrees.
